(19) United States
(12) Patent Application Publication
(10) Pub. No.: US 2009/0299642 A1
(43) Pub. Date: Dec. 3, 2009

(54) METHOD

(75) Inventors: Peter W. French, Sydney (AU); Gary L. Corino, Sydney (AU)

Correspondence Address: MERCHANT & GOULD PC, P.O. BOX 2903, MINNEAPOLIS, MN, 55402-0903, US

(73) Assignee: FERMISCAN AUSTRALIA PTY LIMITED, Sydney, NSW (AU)

(21) Appl. No.: 12/306,130

(22) PCT Filed: Jun. 28, 2007
(86) PCT No.: PCT/AU07/00889

§ 371 Date: Jul. 1, 2009

(30) Foreign Application Priority Data
Jun. 29, 2006 (AU) 2006903509
Jan. 19, 2007 (AU) 2007900288
Jan. 22, 2007 (AU) 2007900306

(51) Int. Cl. G01N 33/48; G06F 19/00

(52) U.S. Cl. 702/19

(57) ABSTRACT

A method of analyzing a keratin sample from a subject to improve sensitivity and specificity of a diagnostic test for a pathological state in the subject by exposing the keratin sample to incident energy derived from an energy source; receiving radiated energy from the keratin sample consequent upon impingement of the incident energy on the keratin sample; passing at least a portion of the radiated energy received from the keratin sample through a transducer so as to derive subject specific data; processing subject specific derived data; comparing the processed subject specific data thus derived with a second group of reference data present in a reference database; wherein the second group of reference data is consistent with a presence of the pathological state in the subject; and including the steps of applying an appropriate algorithm to said subject specific data prior to said step of comparing thereby to improve sensitivity and specificity relative to said reference data.

A method of analyzing a keratin sample from a subject so as to improve sensitivity and specificity of a diagnostic test for a pathological state in the subject comprising exposing the keratin sample to incident energy derived from an energy source; receiving radiated energy from the keratin sample consequent upon impingement of the incident energy on the keratin sample; passing at least a portion of the radiated energy received from the keratin sample through a transducer so as to derive data; processing derived data; comparing the processed data with a second group of data present in a reference database; wherein the second group of data is consistent with a presence of the pathological state in the subject.

### BACKGROUND

Many pathological states may be detectable at a particular stage in their life cycle. However, often this stage can be late in the life cycle of the pathological state thereby producing a poorer prognosis for a subject than would otherwise have occurred had the pathological state been detected at an earlier stage, this situation is particularly noticeable in the case of cancer in which early detection can often significantly improve the chances of subject survival.

Improvements in the sensitivity of a diagnostic test or improvements in specificity in comparison with standard tests can often be associated with reductions in morbidity and mortality in the subject.

By definition sensitivity means the ability of a test to detect the presence of a particular pathological state at a certain stage in the life cycle of the pathological state, for example, a test which detects the presence of a cancer that is not yet invasive is one that is more sensitive than a test which will only reveal the presence of the cancer when the cancer has become invasive. Specificity means the ability of a test to narrow down a possible range of pathological states associated with a given test result; for example a test which implicates one cancer is more specific than one that implicates three possible cancers as being consistent with a positive result for a test. Other tests may be neither specific nor sensitive to the presence of a pathological state and thereby fail to indicate the presence of the pathological state or at best yield an inconclusive result. A test yielding no conclusive results for the presence of a pathological state would be said to have no sensitivity (that is a sensitivity value of zero).

It is therefore an ongoing problem in the treatment of cancers or pathological states in general to improve sensitivity, specificity and conclusiveness of tests for pathological states.

It is an object of the present invention to address or at least ameliorate some of the above disadvantages.

### Notes

1. The term “comprising” (and grammatical variations thereof) is used in this specification in the inclusive sense of “having” or “including”, and not in the exclusive sense of “consisting only of”.
2. The above discussion of the prior art in the Background of the invention, is not an admission that any information discussed therein is citable prior art or part of the common general knowledge of persons skilled in the art in any country.

### BRIEF DESCRIPTION OF INVENTION

In one broad form of the invention there is provided a method of analyzing a keratin sample from a subject so as to improve sensitivity and specificity of a diagnostic test associated with a pathological state in the subject comprising:
a) exposing the keratin sample to incident energy derived from an energy source;
b) receiving radiated energy from the keratin sample consequent upon impingement of the incident energy on the keratin sample;
c) passing at least a portion of the radiated energy received from the keratin sample through a transducer so as to derive data;
d) processing the derived data using the appropriate algorithm
e) comparing the data derived with a second group of data present in a reference database; wherein the second group of data is consistent with a presence of the pathological state in the subject. Preferably, the second group of data is correlated with the presence of the pathological state in the subject.

Preferably, the second group of data is indicative of the presence of the pathological state in the subject.

Preferably, the energy source is selected from a plurality of different energy sources.

Preferably, the keratin sample is selected from a plurality of different keratin samples.

Preferably, the second group of data is selected from a plurality of different data groups of data.

Preferably, the derived data is processed using a plurality of different mathematical methodologies.

Preferably the processed data and the second group of data are analyzed using a plurality of different methods of comparison.

Preferably, at least a portion of the incident energy is absorbed by the keratin sample.

Preferably, in use, the keratin sample can be obtained and analyzed in association with at least one of a pharmacy, a test kit, the subject's home, a health care clinic and a testing laboratory.

In a further broad form of the invention there is provided a method of analysis of the data derived in the method as described above wherein said data is in the form of image data of an image derived from said transducer; said method of analysis comprising:
(a) extracting one-dimensional data along predetermined paths in said image so as to determine spacing of features in said image
(b) defining substantially circular-oriented peak data about a centre point of said image from an analysis of said one dimensional data
(c) applying intensity correction to said substantially circular-oriented peak data so as to better define said circular-oriented peak data as it appears in said image.

### BRIEF DESCRIPTION OF DRAWINGS

FIG. 1 shows a keratin sample being exposed to incident radiation.

FIG. 2 shows a plurality of different types of incident radiation directed upon a keratin sample.

FIG. 3 shows a plurality of different keratin samples being exposed to incident energy from a given energy source.

FIG. 4 shows a plurality of different methods of analysis being used to analyze data produced according to the first embodiment disclosed in FIG. 1.

FIG. 5 shows a keratin sample being exposed to incident radiation that is being partially or completely diffracted in association with the method disclosed in FIG. 1.

FIG. 6 shows a keratin sample partially or completely absorbing incident radiation in association with the method of analyzing a keratin sample disclosed in FIG. 1.

FIG. 7 shows the method of analyzing a keratin sample being implemented in use, wherein a sample from a subject can be collected by an appropriate professional at a collecting room or conduct a test using a kit at a convenient location.

FIG. 8 is a block diagram of a beam line layout and main processing steps in accordance with the embodiments of the present invention,

FIG. 9 is an example of a one dimensional data plot for three different sectors as required in one of the processing steps of FIG. 8.

FIG. 10 is a comparison of images derived from two different image processing protocols.

FIG. 11 illustrates x-ray diffraction patterns derived from embodiment methods of the present invention showing for comparison patterns derived from “normal” hair; a “disordered” pattern from a defective sample and a pattern from hair indicative of breast cancer.

FIG. 12 illustrates multiple patterns representing reproducibility.

FIG. 13 illustrates comparative patterns resulting from following the enhanced method in accordance with embodiments of the present invention.

FIG. 14 shows an example of the method in use for detection of disease in an animal (Tasmanian Devil).

### DETAILED DESCRIPTION OF PREFERRED EMBODIMENTS

### Definitions

“Radiate”: To proceed in direct lines from a point or surface.
“Mammalian species” includes the types of species as appearing in the body of the specification. It can include a human, a pet such as a dog or cat or a variety of other animals.
“Energy source” includes the types of energy as appearing in the body of the specification.
A “keratin sample” or a “keratin substance” is a sample that is substantially comprised of keratin. The keratin sample or substance can include human scalp or body hair and in particular pubic hair, pet hair, animal hair or hair from a mammalian species in general, or other keratin based materials such as nail clippings or an eyelash.
A “subject” is an individual member of a mammalian species. A mammalian species can include a human, a pet such as a dog or cat, an agricultural animal such as a cow, and any other animal with hair.

The plurality of different selections and forms pertaining to the invention as claimed include the selections and forms as appearing in the body of the specification.

Unless otherwise indicated by the context, a claim to one element is consistent with a claim to at least one element.

Embodiments of the present invention will now be described with reference to the accompanying drawings wherein:

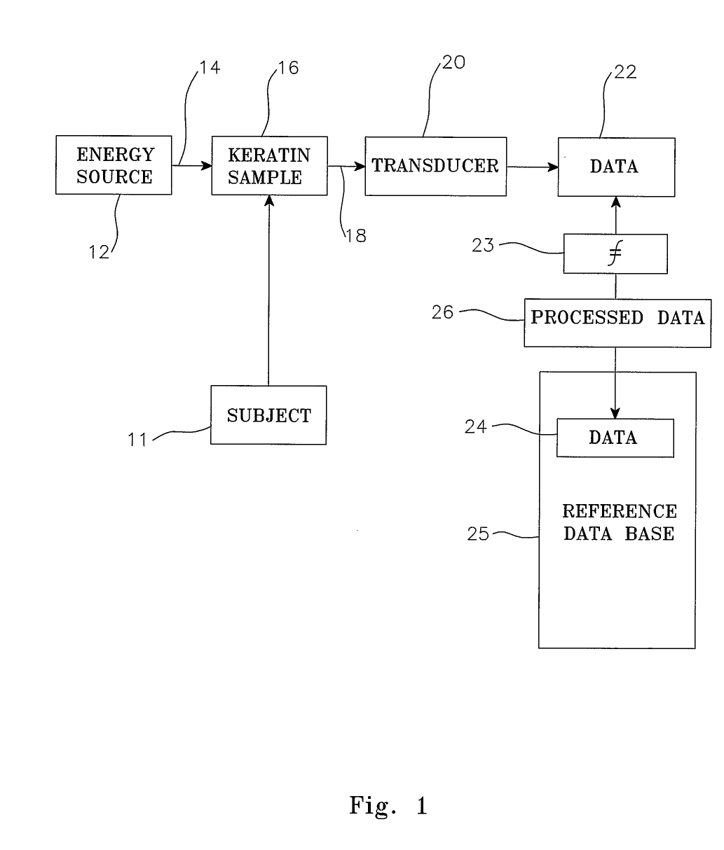

FIG. 1 illustrates a method of analyzing a keratin sample 16. FIG. 1 shows an energy source 12 from which incident energy 14 emanates. A keratin sample 16 is taken from subject 11. The subject 11, includes any member of a mammalian species. A mammalian species can include a human, a pet such as a dog or cat or and other animal with hair. The keratin sample 16 can include human scalp or body hair and in particular pubic hair, pet hair, animal hair or hair from a mammalian species in general. The hair sample can be either a single hair fiber or multiple hair fibers from the same subject. Other keratin-based materials such as nail clippings, claws, hooves, skin or an eyelash can be used.

In a particular form the hair making up a single sample comprises up to five strands. In further particular forms the bundle of strands is placed in a capillary tube.

The keratin sample 16 is exposed to the incident energy 14 derived from energy source 12. Radiated energy 18 is derived from the keratin sample 16 consequent upon impingement of the incident energy 14 on the keratin sample 16. At least a portion of the radiated energy 18 is passed through a transducer 20 to produce data 22.

The data 22 is then processed in a processing step 23 to produce processed data 26 using an appropriate algorithm which filters, averages and subtracts the data.

As will be described further in the specification in preferred forms the processing step can include smoothing of the raw data and subtraction of a background image from the smoothed image in order to (although not exclusively) remove rising intensity data at lower values of Q.

The processed data 26 can be compared with data 24 in a reference database 25 to determine whether or not the subject 11 may have a pathological state (for example if the reference database 25 indicates that the result in question correlates with the presence of the pathological state then a meaningful comparison can be considered. Additionally zero correlation can also provide useful analytical information).

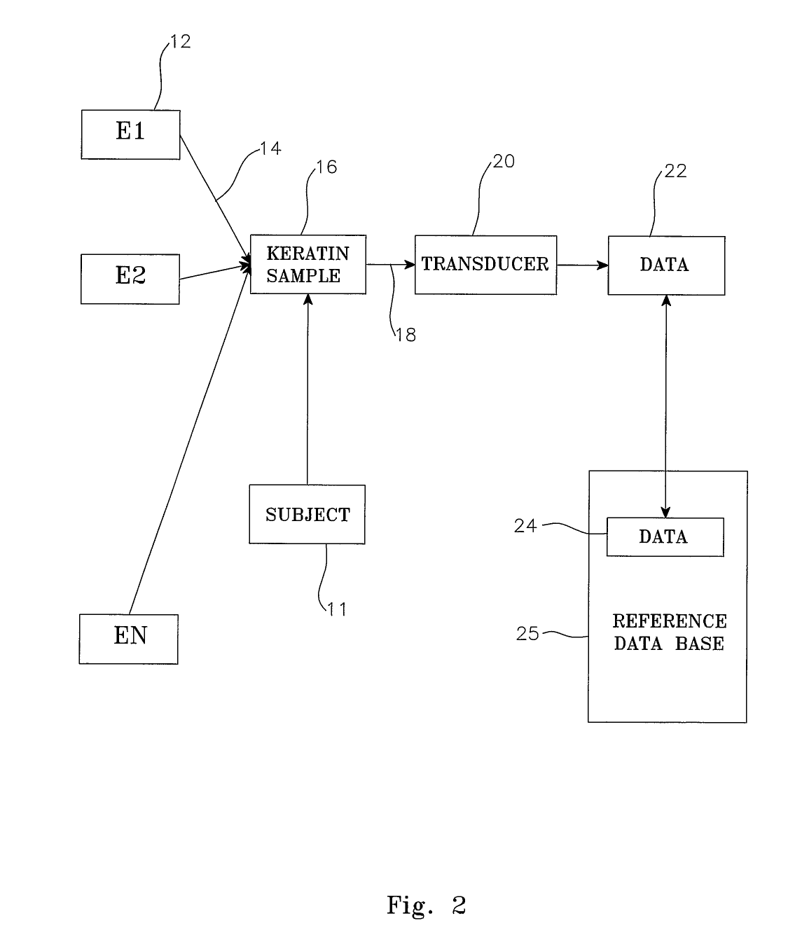

FIG. 2 shows an embodiment of the present invention in which the sensitivity or specificity of the method described in FIG. 1 is improved by way of changing the energy source 12. FIG. 2 shows a plurality of different energy sources 12, being E1, E2 . . . EN, which are shown to produce different types of incident energy 14.

In FIG. 2 the data 22 is analyzed by comparing the data with data 24 in the reference database 25 so as to select an energy source from the set E1. EN, which is adapted to lead to an improvement in sensitivity or specificity of the method of analyzing a keratin sample.

The energy sources in FIG. 2 can include different types of electromagnetic radiation. Alternatively or additionally the same type of electromagnetic radiation can be chosen such as energy in the visible light spectrum but a new availability can be sought in relation to the same type of energy source 12. By a new availability it is meant a hitherto unknown property of the known type of electromagnetic radiation which can yield greater specificity or sensitivity of the method in association with a given keratin sample 16.

The wavelength or frequency of the electromagnetic radiation can be altered without removing the incident energy 14 from the visible spectrum of light for example (a new availability can be sought within a specific sub range of visible light that can lead to greater specificity or sensitivity). A variation in the frequency, amplitude, wavelength or other availabilities of the energy source 12 could lead to an improvement in the sensitivity or the specificity of the method of analyzing the keratin sample 16. For example if blue light could yield greater sensitivity of the method than include sound waves. Further, the energy source 12 can include one or more of the following types of energy sources being infrared radiation, UV radiation, Raman energy, laser radiation or X-Ray radiation without limitation.

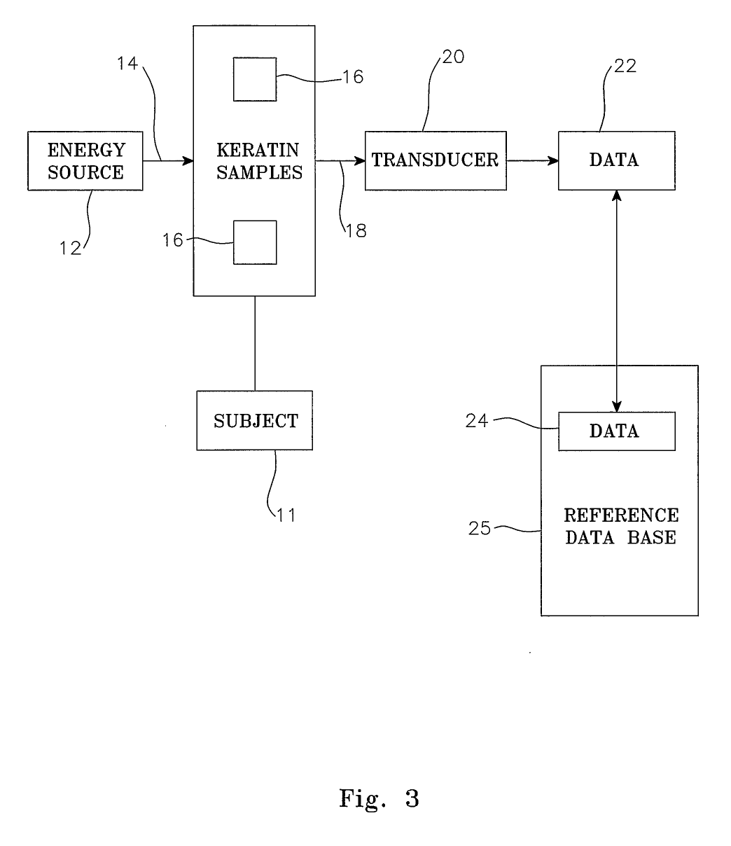

FIG. 3 shows the use of a specific energy source 12, for a given mode of operation in association with a plurality of different keratin samples 16. The plurality of different keratin samples 16 can be taken from a plurality of different mammalian species (two have been shown but more can be included). Alternatively or additionally, the plurality of different keratin samples 16 can be taken from subjects who are suspected to have a plurality of different pathological states.

The method of analyzing a keratin sample 16 described in FIG. 1 is conducted again in the embodiment shown in FIG. 3 so as to select a particular keratin sample 16 which demonstrates a particular susceptibility to the incident radiation 14 in association with the method, which thereby leads to an improvement in sensitivity or specificity of the method of analysis. A particular susceptibility will occur if a keratin sample is known to yield an improvement in either specificity or sensitivity of the method in association with a given mode of operation and a given energy source 12.

The plurality of different keratin samples 16, shown in FIG. 3 can include a keratin sample 16 taken from a subject 11 who is suspected of having a pathological state which can include one or more cancers or pathological states such as lung cancer, Creutzfeldt-Jacob disease, mad cow disease, infection (bacterial, or a prion or more generally by other infectious agents), a metabolic disorder (which can include diabetes, or alternatively hepatitis), heart disease or liver dysfunction. Further, without limitation the keratin sample 16 can include keratin Type I and keratin Type II.

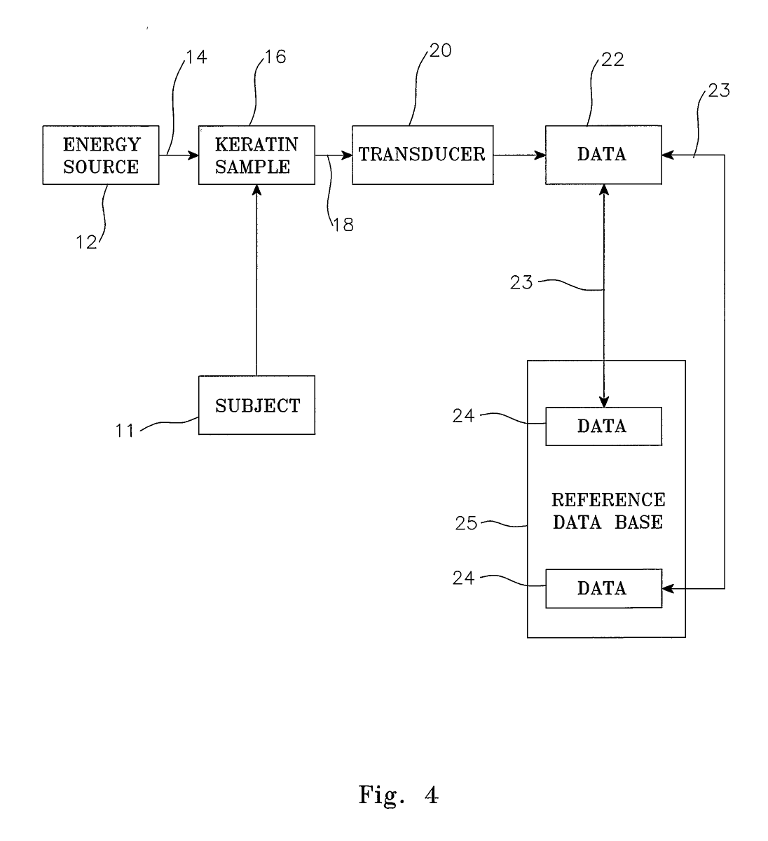

FIG. 4 shows an embodiment of the method of analyzing a keratin sample 16 as shown in FIG. 1 in which a given energy source 12 is used in association with a given keratin sample 16 together with a plurality of different types of methods of comparison 23 between the data 22 and data 24 so as to produce an improvement in specificity or sensitivity of the method of analyzing the keratin sample 16. The plurality of different comparisons 23 shown in FIG. 4 can without limitation (two have been shown but more can be included) include one or more of the following variations in the mode of operation of the method of analyzing a keratin sample 16 including spectral analysis or the use of pattern recognition computer programs. The plurality of different comparisons 23 can include a comparison to detect different patterns in relation to X-ray diffraction (for example a complete ring at Q=1.32+/−0.02 nm¹) that can be consistent with the presence of breast cancer.

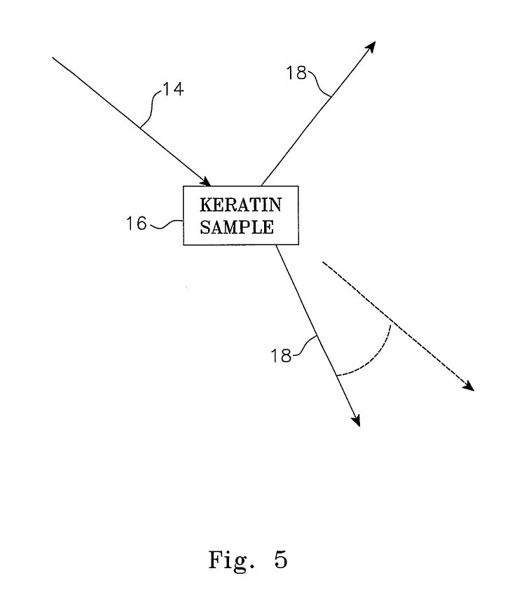

FIG. 5 shows radiated energy 18 that is not necessarily completely reflected from the keratin sample 16. Rather the radiated energy 18 can be partly or completely diffracted.

The data 22 derived from the radiated energy 18, which is diffracted can be analyzed using the method of analyzing the keratin sample 16 shown in FIG. 1 so as to produce improvements in the specificity or sensitivity of the method of analyzing the keratin sample 16.

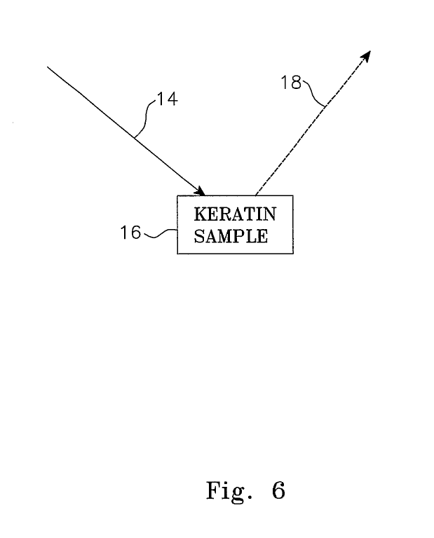

FIG. 6 shows incident radiation 14 that can be partially or completely absorbed. The degree of absorption can produce derived data 22, or an absence of data 22, which can be consistent with the presence of a pathological state in the subject 11. The derived data 22, associated with the embodiment as seen in FIG. 6 can be used to improve the sensitivity or specificity of the method of analyzing a keratin sample disclosed in FIG. 1.

### In Use

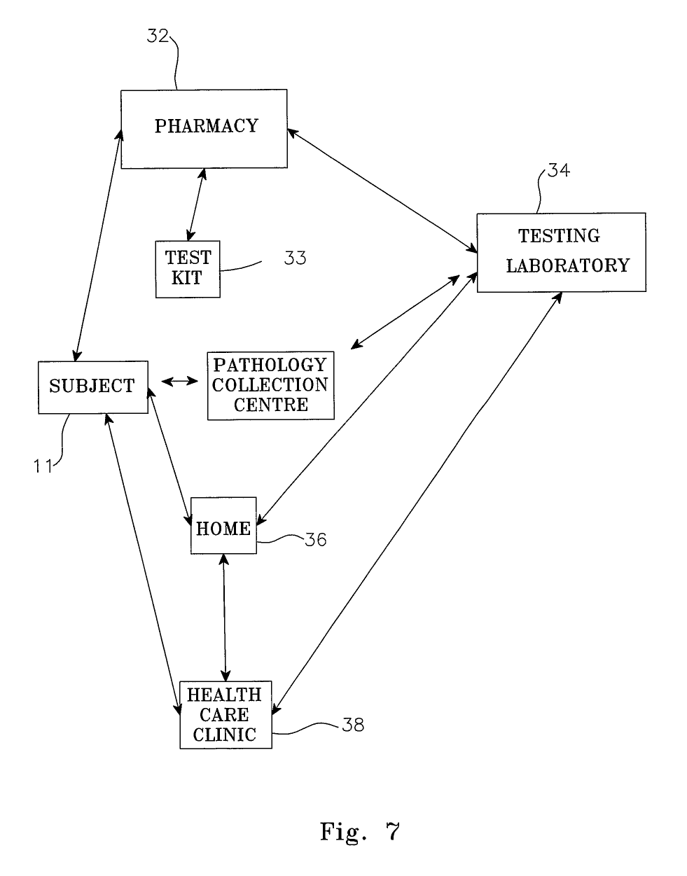

FIG. 7 shows an embodiment of the present invention in use.

In FIG. 7 a subject 11 can attend a pharmacy 32 to provide a keratin sample 16. The keratin sample 16 can then be sent to a testing laboratory 34 so as to perform the method of analyzing the keratin sample 16 as seen in FIG. 1.

Additionally, a test kit 33 can be obtained so as to collect the keratin sample 16 from the subject at a convenient location (e.g. home, office, field or barn), said sample can be sent to test testing laboratory 34 so as to perform the method of analyzing the keratin sample 16 as seen in FIG. 1.

Alternatively, the subject 11 can attend a health care or veterinary clinic 38 so as to provide the keratin sample 16. The clinic 38 can perform the method of analyzing the keratin sample 16 or forward the keratin sample to the testing laboratory 34.

### Further Embodiment

A preferred image analysis method has been trialed and is described below:

### Sample Collection and Handling

Hair samples (scalp and/or pubic) of at least 30 mm in length were collected from women referred to an Australian radiology clinic for a mammogram. Women were excluded if their scalp hair had been dyed or chemically treated (such as permanent waving) within the previous 6 weeks and if their pubic hair was unavailable, or had a history of breast cancer or other cancers (excluding non-melanoma skin cancer and CIN: cervical intra-epithelial neoplasia within 5 years. Nineteen blinded hair samples were collected at the clinic and these samples together with 14 samples from women diagnosed with breast cancer and six samples from women assumed negative by mammography, were analysed in this study.

Scalp hairs were taken from the region behind the ear, close 5 to the hair line, and removed by cutting as close to the skin as possible. This was done to ensure the samples taken had minimal damage from environmental factors. Pubic hairs were also removed by cutting as close to the skin as possible and all hair samples were stored in plastic specimen containers.

All subject medical histories were kept on file at the clinic.

Synchrotron Small Angle X-Ray Scatter (SAXS) analysis required a single hair to be gently removed from the container using fine forceps and loading it onto a specially designed sample holder that is capable of holding individual hair fibers. These holders use fine springs to grasp a fiber and pins to locate the fibre in the appropriate orientation for the X-ray beam. When it could be identified, the cut end of the fiber was loaded first by opening the coils of a spring on one side of the holder and placing the fiber between the coils. The spring was then allowed to relax to clamp the fiber. The coils of the spring opposite were then opened and the loose end of the fiber was inserted into the coils. The hair was placed adjacent to the locating pins then the spring was gently released. A great deal of care was taken with the loading process to ensure the fiber was not twisted during loading or that it was not damaged by stretching. Once loaded, the hairs were examined under a dissecting stereo microscope.

### X-Ray Diffraction

Synchrotron SAXS experiments were carried out at the Advanced Photon Source at the Argonne National Laboratory, USA.

Analyses were conducted using the beamlines 18-ID (BioCAT) and 15-ID (ChemMatCARS).

The beam characteristic for the BioCAT experiment was 70 microns in the vertical and 200 microns in the horizontal and a wavelength A=1.03 Angstroms. The hairs were mounted with the axis of the hair in the parallel plane and at a zero angle of incidence. The sample's optimal position in the beam was determined by use of a CCD detector (Aviex Electronics, USA). The fiber was exposed to X-rays for 2 seconds and the diffraction image assessed for characteristic features that indicate if the fiber is centrally located in the beam. Once optimally located, the fiber was exposed to X-rays for approximately 20 seconds and the diffraction image collected on Fuji BAS I11 image plates that had an active area of approximately 190 mm×240 mm. The space between the sample and detector was held under vacuum to reduce air scattering, and this distance was determined to be 959.4 mm by analysis of the scattering pattern of Silver Behenate.

The beam characteristic used for the ChemMatCARS experiment was 300 micrometres in the vertical and 500 micrometres in the horizontal and the wavelength used was A=1.50 Angstroms. This translated to lower beam flux at the sample and hence longer sample exposure tines but it facilitated sample positioning as the hair was fully encompassed within the X-ray beam. Hair samples were exposed to the X-ray source for 60 seconds and the diffraction images were collected on a MAR345 detector. The space between the sample and detector was held under vacuum to reduce air scattering, and this distance was determined to be 635.8 mm by analysis of the scattering pattern of Silver Behenate.

### Image Analysis

Diffraction images were analysed using FIT2D and Saxsl5ID software packages. Both programs offer the data manipulation and smoothing routines that are required to perform the data reduction and subsequent analysis. Extracted one dimensional data from these packages was visualized and analysed using the Spectrum Viewer software package.

Two methods and parameters were employed to enhance the SAXS image by smoothing and subsequent background removal. The first one, which we hereinafter call the “Standard Protocol”, is known to have only been described in one publication by James (Reference: Wilk K, James V, and Amemiya Y. Intermediate Filament Structure of Human Hair. Biophysica Biochimica Acta. 1995; 1245: 392-396). In no publication by James does she describe the complete recipe of how to process the raw SAXS data and the parameters used to detect the presence of cancer. No previous publication contains a complete method that could be used by an independent observer to determine the incidence of breast cancer from a SAXS image. Whether or not the parameters and methods used to process the SAXS images by James have been developed since first published is unknown, but a clear and concise description of the complete method to process the SAXS images 20 to diagnose breast cancer remains unpublished. In brief, smoothing the raw SAXS image is achieved by replacing the value of the central pixel of a 3 by 3 box of pixels with the average value calculated over that box. A background image is created by blurring the smoothed image in a similar manner to that described above but with a 20 by 20 box of pixels. The image used for the diagnosis of breast cancer is produced by subtracting the created background image from the smoothed image. The purpose of background correction is to remove the rising intensity at lower values of Q without compromising any of the features present in the original image. FIT2D has two different smoothing functions available to the user, “Smooth” and “Median”.

In the course of this study we developed an alternative background correction protocol to attempt to smooth the raw data and to produce a background image that, when subtracted from the smoothed data, did not remove or occlude important features which were present at low intensity in the original image. The SAXS images were initially smoothed using a 3 by 3 pixel “median” filtering operation, which allows smoothing without loss of subtle features, followed by a 50 by 50 pixel “averaging” to create a background from the smoothed image. We refer to this as the “Alternative Protocol”.

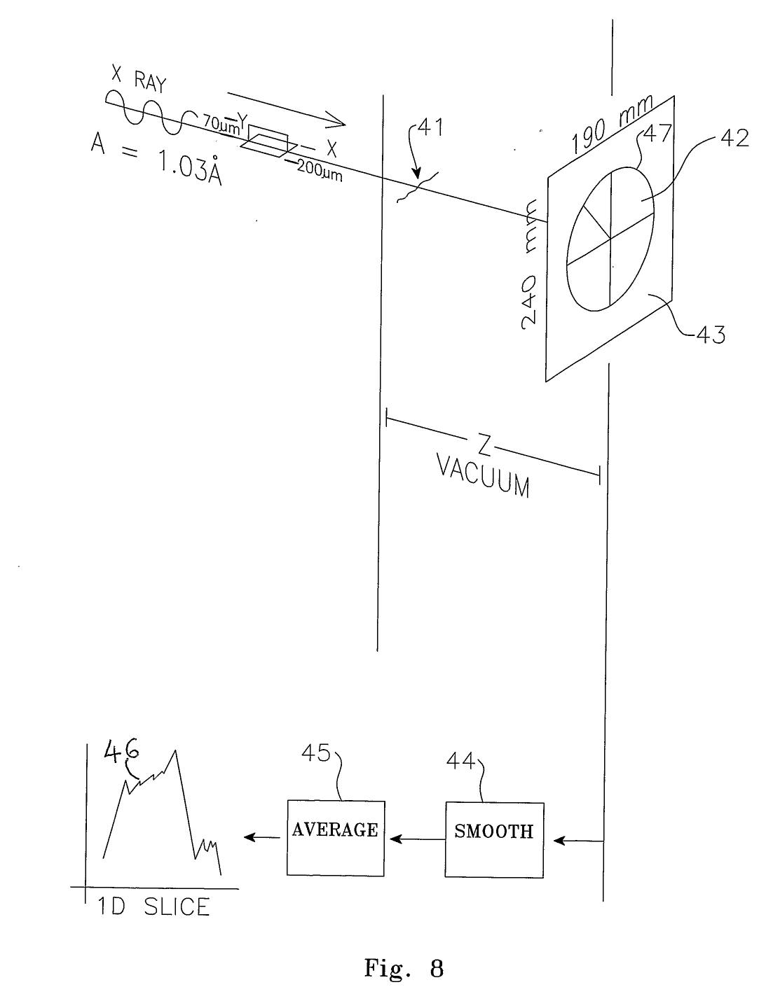

With reference to FIG. 8 a typical beam line layout together with image processing steps in accordance with a preferred embodiment of the invention is illustrated in block form.

In this instance an X ray beam 40 having wavelength A and having width X and height Y is directed at hair sample 41 with the resulting diffraction image 42 appearing on plate 43 at distance Z from sample 41. In a particularly preferred form the volume defined between sample 41 and plate 43 is maintained in at least a partial vacuum to minimize scatter.

The diffraction image 42 is digitized and a soothing algorithm 44 is applied followed by an averaging algorithm 45. One dimensional slices 46 are then taken along selected sectors 47 derived from the original diffraction image in the plane of plate 43.

In one particular non limiting form A 1.03 Angstroms, X=200 microns, Y=70 microns and Z=959 mm.

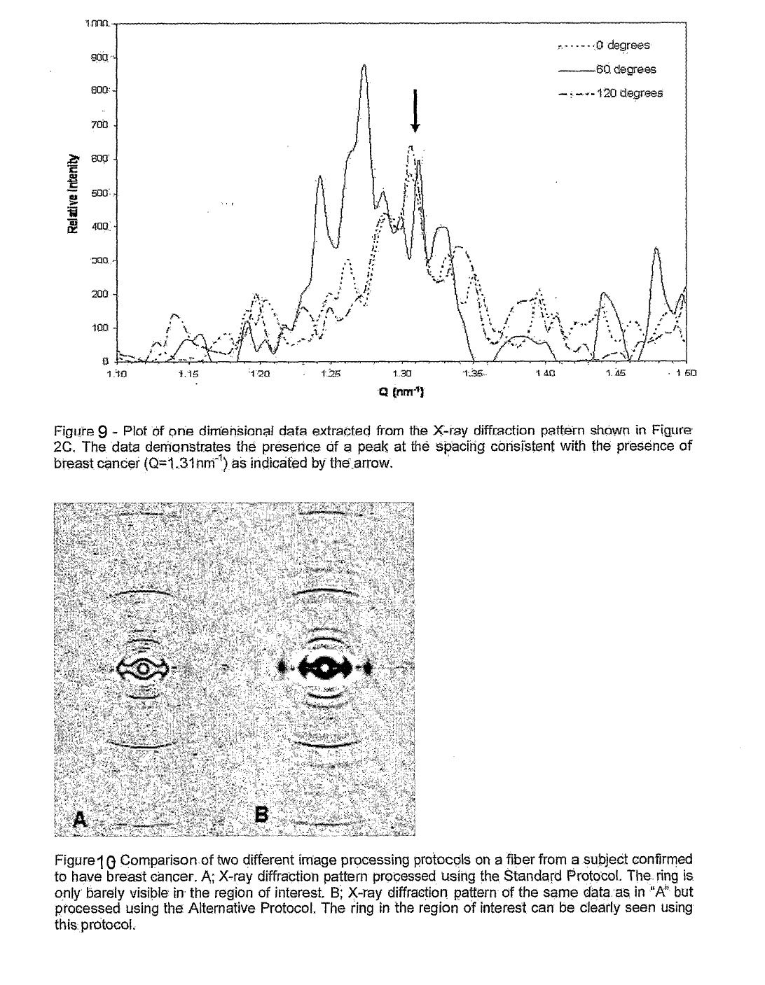

One-dimensional data for example as shown in FIG. 9 was extracted from each SAXS image to determine the exact spacing of features in the image. This was achieved by two different methods. The first was to extract the intensity data along a single line starting from the centre of the image along the meridional plane at 0°, 60°, 120°, 180°, 240° and 300°. This process was used to ensure that if a ring was present in the SAXS image, the intensity data would show a peak in the appropriate location and from the analysis of the data from all four quadrants its circular nature could be established.

For SAXS images that demonstrated weak features at the approximate spacing of the ring indicative to the presence of breast cancer, a modification to the method of data extraction described above was used. In these cases intensity data was extracted by integrating 5° sectors at the locations to the meridional mentioned above. This was performed in an attempt to increase the level of signal over background noise of weak data.

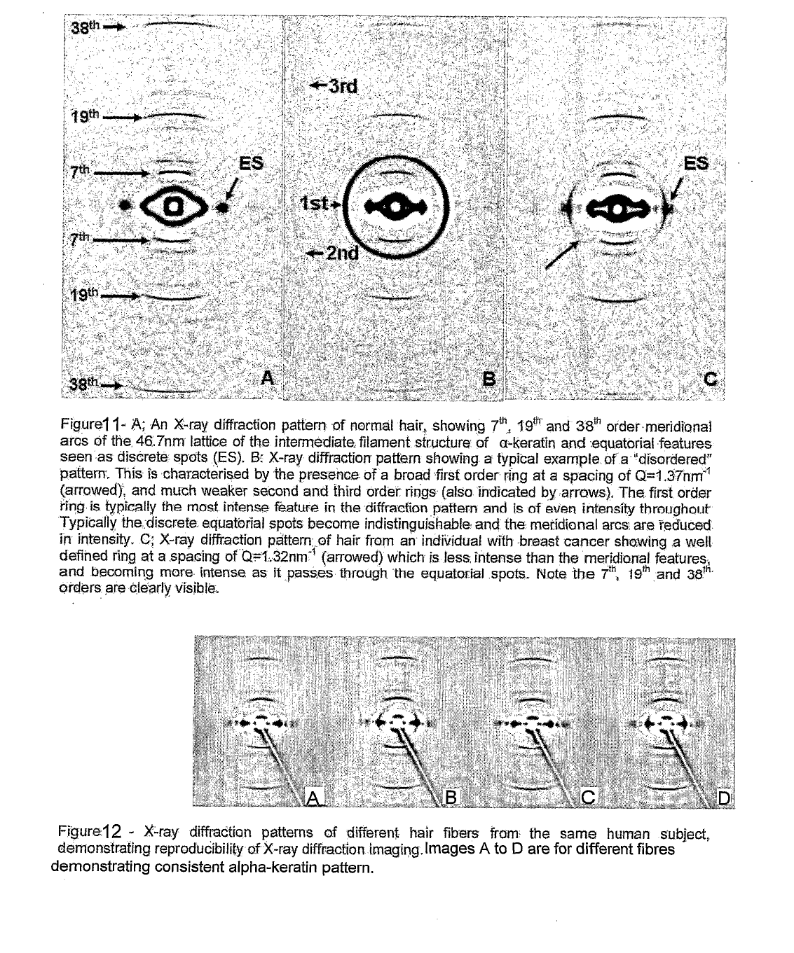

With reference to FIG. 10:

### Defining the Breast Cancer SAXS Pattern—First Trial

Using the Standard Protocol for image processing we were able to identify the ring correlating to the presence of breast cancer in 13 of the 14 positive controls at the defined spacing (Q=1.33 nm⁻¹; more preferably Q=1.32+/−0.02 nm⁻¹).

None of the samples assumed negative by mammography demonstrated a ring at that spacing in their respective SAXS patterns. One-dimensional data extracted from the respective SAXS patterns confirmed the above findings. The Standard Protocol was then used to assess the blinded samples that were collected at the radiology clinic. The subject's pathology and results of the analyses using the Standard Protocol are shown in Table 1. From the information presented in the Table it can be seen that only 1 of the 19 samples collected came from a woman with confirmed breast cancer. Analysis of the SAXS pattern for this particular sample using the Standard Protocol produced an image with only a very faint and slightly elliptical ring in the zone of interest. One-dimensional data extracted from this image indicated the presence of a ring but was not significant above the background and was therefore designated as negative. After the samples were unblinded, this result was classified as a false negative. Of the other samples, three showed a ring in the zone of interest and were designated positive and another showed a ring in the zone of interest and also displayed evidence of disorder but was still designated positive. The other samples were declared negative.

From the SAXS analysis results generated using the Standard Protocol, it was apparent that the disclosed methodology and parameters used by James for image processing were not suited to images that contain weak and/or diffuse features. We subsequently re-analysed the images using the Alternative Protocol of data reduction to ensure that faint but significant information in the area of interest was not lost as a result of image processing.

Using Fit2D and Saxl5ID with the Alternative Protocol, the positive control samples were reassessed. From these results, and the extracted one dimensional data, we determined the spacing of the ring correlating to the presence of breast cancer to be Q=1.32+/−0.02 nm⁻¹. The mean+/−2 SDs was applied as the key quantitative criterion to define the zone of interest. Use of the Alternative Protocol produced superior and more detailed SAXS images compared to those of the Standard Protocol. FIGS. 9A and 9B are the resultant images from applying the Standard Protocol and the Alternative Protocol respectively to the sample designated negative and later classified as a false negative. As can be seen in FIG. 9B, a weak diffuse ring can now be seen. The one dimensional data extracted from this image defined the ring to have an approximate spacing of Q=1.32±0.02 nm⁻¹ (d=4.76+/−0.07 nm). Thus the Alternative Protocol of image reduction produced superior data more particularly where diffuse low intensity information was observed.

TABLE 1
Comparison of SAXS data with mammography results in a set of subjects attending a radiology clinic

| | | | Standard Protocol | Alternative Protocol |
|---|---|---|---|---|
| Code # | Clinic procedure | Patient notes | (blinded analysis) | (unblinded) |
| 40761 | Biopsy | Negative | Negative | Negative |
| | | Calcium oxalate | (ring at 0.137) | |
| 248057 | Mammography/ | Negative | Negative | Negative |
| | ultrasound/Biopsy | Benign breast tissue | (no ring) | (no ring) |
| 594776 | Biopsy | Positive | Query Positive | Positive |
| | | Infiltrating carcinoma | (faint ring 0.130) | (ring at 0.130) |
| 631895 | Mammography/ | Negative | Negative | Negative |
| | ultrasound | | (no ring) | (no ring) |
| 664921 | Mammography/ | Negative | Negative | Negative |
| | ultrasound | | (no ring) | (no ring) |
| 966848 | Mammography/ | Negative | Negative | Negative |
| | ultrasound | Cysts | (no ring) | |
| 6169711 | Ultrasound | Negative | Disorder, | Disorder |
| | | 3 mm Cyst | Query positive | |
| 9007130 | Mammography/ | Negative | Negative | Negative |
| | ultrasound | Fibroadenoma | (ring at 0.138) | (ring at 0.137) |
| 9008728 | Mammography | Negative | Negative | Negative |
| | | | Disordered (no ring) | (no ring) |
| 9025794 | Mammography/ | Negative | Positive | Positive |
| | ultrasound | | (faint ring at 0.133) | (ring at 0.132) |
| 9030217 | Mammography/ | Negative | Negative | Disorder |
| | ultrasound | Calcific foci | (strong ring at 0.130) | |
| 9033550 | Mammography | Negative | Disorder | Disorder |
| | | Mammary implants | (ring at 0.130) | Ring at 0.130 + orders |
| 9039174 | Mammography/ | Negative | Negative | Negative |
| | ultrasound | Multiple cysts | (very faint/no ring) | (no ring) |
| 9076831 | Mammography | Negative | Positive | Positive |
| | | Post-surgical deformity | (faint ring at 0.133) | (faint ring at 0.132) |
| | | and benign calcific foci | | |
| 9079870 | Ultrasound | Negative | Negative | Negative |
| | | Cyst | (non-continuous | (Faint non-continuous |
| | | | feature at 0.140) | diffuse feature at 0.129) |
| 9085332 | Mammography/ | Negative | Disorder | Disorder |
| | ultrasound | | | |
| 9091902 | Mammography/ | Negative | Negative | Negative |
| | ultrasound | | (no ring) | (no ring) |
| 9126804 | Mammography/ | Negative | Positive | Positive |
| | ultrasound | Post-surgical deformity | (ring at 0.133) | Ring at 0.132 with some |
| | | and multiple cysts | | disorder |
| 9235226 | Mammography | Negative | Disorder | Disorder |
| | | Probable cysts | | |

### Defining the Breast Cancer SAXS Pattern—Second Trial

### Sample Collection and Handling

72 hair samples (scalp and/or pubic) were collected from women referred to an Australian radiology clinic for a mammogram. Ethics approvals regarding sample collection and subject information were sought and granted by a registered ethics committee. Women were able to supply hair samples if they were willing and able to provide informed consent and had usable scalp or pubic hair at least 30 mm in length. Women were excluded if their scalp hair had been dyed or chemically treated (such as permanent waving) within the previous 6 weeks and if their pubic hair was unavailable, or had a history of breast cancer or other cancers (excluding non-melanoma skin cancer and CIN: cervical intra-epithelial neoplasia) within 5 years. Nineteen hair samples had been collected by the clinic at the time the synchrotron was accessed, and these samples together with 14 samples from women diagnosed with breast cancer and six samples from women assumed negative by mammography, were analysed in this study.

Scalp hairs were taken from the region behind the ear, close to the hair line, and removed by cutting as close to the skin as possible. This was done to ensure the samples taken had minimal damage from environmental factors. Pubic hairs were also removed by cutting as close to the skin as possible and all hair samples were stored in plastic specimen containers.

Samples were coded at the radiology clinic with a unique identifying number and supplied “blinded” as they had no other identifying information other than that code.

All subject medical histories were kept on file at the clinic. A single hair was gently removed from the container using fine forceps and loaded onto a specially designed sample holder that is capable of holding 10 individual hair fibers

### (FIG. 1).

Synchrotron SAXS experiments were carried out at the Advanced Photon Source at the Argonne National Laboratory, USA.

Analyses were conducted using the 15-ID (ChemMatCARS) beamline.

15-ID (ChemMatCARS) has optics that allows focusing in one direction only. The beam size used for the experiments on this beam line was 300 microns in the vertical and 500 microns in the horizontal and defined by the use of slits. The wavelength used was 0.150 nm. Hair samples were exposed to the X-ray source for 60 seconds and the diffraction images were collected on a MAR345 detector. The space between the sample and detector was held under vacuum and the distance from sample to detector was determined to be 635.8 mm using the calibration standard mentioned above.

### Image Analysis

Diffraction images were analysed using FIT2D software (FIT2D is described as a general purpose and specialist 1 and 2 dimensional data analysis program, available from and used on the European Synchrotron Research Facility beam-lines) and Saxsl5ID software (refer Cookson, D J (2005) “Saxsl5ID Software for acquiring, processing and viewing SAXS/WAXS image data at ChemMatCARS”).

Both programs offer the data manipulation and smoothing routines that are required to perform the data reduction and subsequent analysis. In previous studies by James, SAXS images were analysed using the astronomy software packages IRAF and SAO, though James has noted that similar results have been obtained by associates using FIT2D software. (refer James V, Corino G Robertson T, Dutton N, Halas D, Boyd A, Bentel J, Papadimitriou J. Early diagnosis of breast cancer by hair diffraction. Int J Cancer. 2005; 114:969-972.)

Extracted one dimensional data from these packages was visualized and analysed using Spectrum Viewer (Spectrum viewer: reads and displays XY-plots on an XY-graph or as a 2D intensity plot It is available from the Department of Applied Physics at Eindhoven Technical University in The Netherlands.

The ‘Alternative Protocol’ was employed to enhance the SAXS image by smoothing and subsequent background removal. The SAXS images were initially smoothed using a 3 by 3 pixel “median” filtering operation, which allows smoothing without loss of subtle features, followed by a 50 by 50 pixel “averaging” to create a background from the smoothed image.

One-dimensional data was extracted from each SAXS image to determine the exact spacing of features in the image. This was achieved by two different methods. The first was to extract the intensity data along a single line starting from the centre of the image along the meridional plane at 0°, 60°, 120°, 180°, 240° and 300°. This process was used to ensure that if a ring was present in the SAXS image, the intensity data would show a peak in the appropriate location and from the analysis of the data from all four quadrants its circular nature could be established. For SAXS images that demonstrated weak features at the approximate spacing of the ring indicative to the presence of breast cancer, a modification to the method of data extraction described above was used. In these cases intensity data was extracted by integrating 50 sectors at the locations to the meridional mentioned above. This was performed in an attempt to increase the level of signal over background noise of weak data.

### Results

19 samples were positive by mammography, and went on to have biopsies performed. Of these 19, 10 were found to be negative for breast cancer by pathology. The hair test picked 9 of these 10 as negative. There was therefore 1 false positive.

Of the 9 cases that were found to be confirmed invasive cancer, 6 were positive using hair x-ray diffraction, and 3 were missed. It was subsequently found that these samples had evidence of being permanently dyed, with very little re-growth.

| | Mammo+ | Mammo+ | |
|---|---|---|---|
| | Biopsy+ | Biopsy− | Mammo− |
| Hair+ | 6 | 1 | 13 |
| Hair− | 3* | 9 | 59 |
| TOTAL | 9 | 10 | 72 |

There were 13 samples that tested positive using x-ray diffraction of hair but were assumed to be negative for breast cancer by mammography.

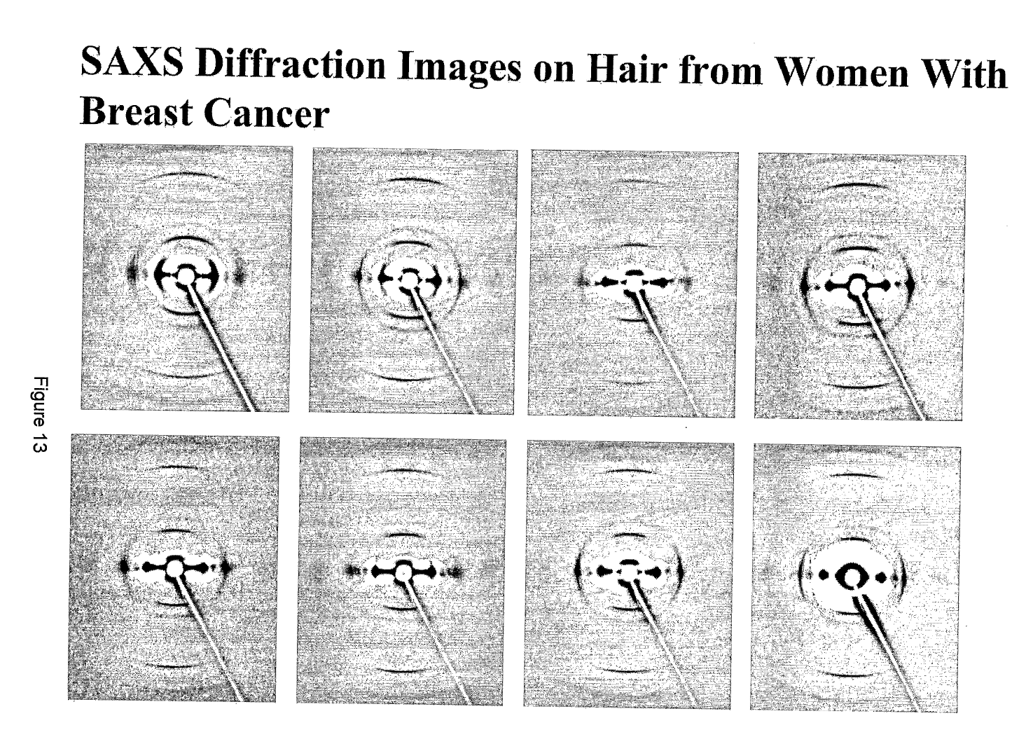

FIGS. 11, 12, 13 illustrate typical diffraction patterns derived using the above described methodology.

FIG. 9 is a plot of one dimensional data extracted from the X-ray diffraction pattern shown in FIG. 11C. The data demonstrates the presence of a peak at the spacing consistent with the presence of breast cancer (Q=1.31 nm⁻¹) as indicated by the arrow.

FIG. 10 is a comparison of two different image processing protocols on a fiber from a subject confirmed to have breast cancer.

FIG. 10A is an X-ray diffraction pattern processed using the Standard Protocol. The ring is only barely visible in the region of interest.

FIG. 10B is an X-ray diffraction pattern of the same data 10 as in “A” but processed using the Alternative Protocol. The ring in the region of interest can be clearly seen using this protocol.

FIG. 11A is an X-ray diffraction pattern of normal hair, 15 showing 7th, 19th and 38th order meridional arcs of the 46.7 nm lattice of the intermediate filament structure of alpha keratin and equatorial features seen as discrete spots (ES).

FIG. 11B is an X-ray diffraction pattern showing a typical example of a “disordered” pattern. This is characterised by the presence of a broad first order ring at a spacing of Q=1.37 nm⁻¹(arrowed), and much weaker second and third order rings (also indicated by arrows). The first order ring is typically the most intense feature in the diffraction pattern and is of even intensity throughout. Typically the discrete equatorial spots become indistinguishable and the meridional arcs are reduced in intensity.

FIG. 11C is an X-ray diffraction pattern of hair from an individual with breast cancer showing a well defined ring at a spacing of Q=1.32 nm⁻¹(arrowed) which is less intense than the meridional features, and becoming more intense as it passes through the equatorial spots. Note the 7th, 19th and 38th orders are clearly visible.

FIG. 12 shows X-ray diffraction patterns of different hair fibers from the same human subject, demonstrating reproducibility of X-ray diffraction imaging.

FIG. 12A to 12D are patterns derived from different fibers demonstrating consistent alpha-keratin pattern.

FIG. 13 illustrates comparative patterns resulting, from following the enhanced method in accordance with embodiments of the present invention.

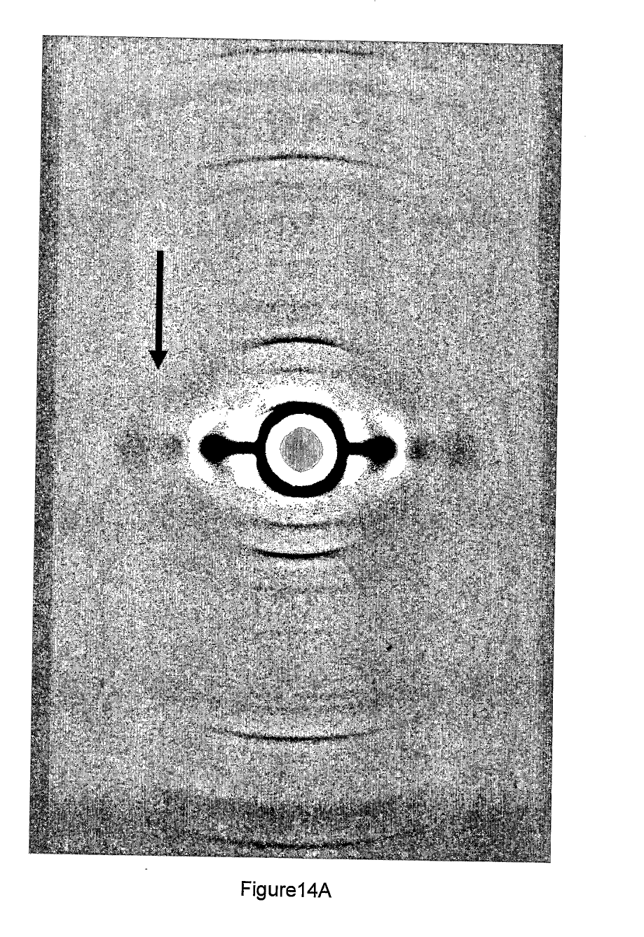

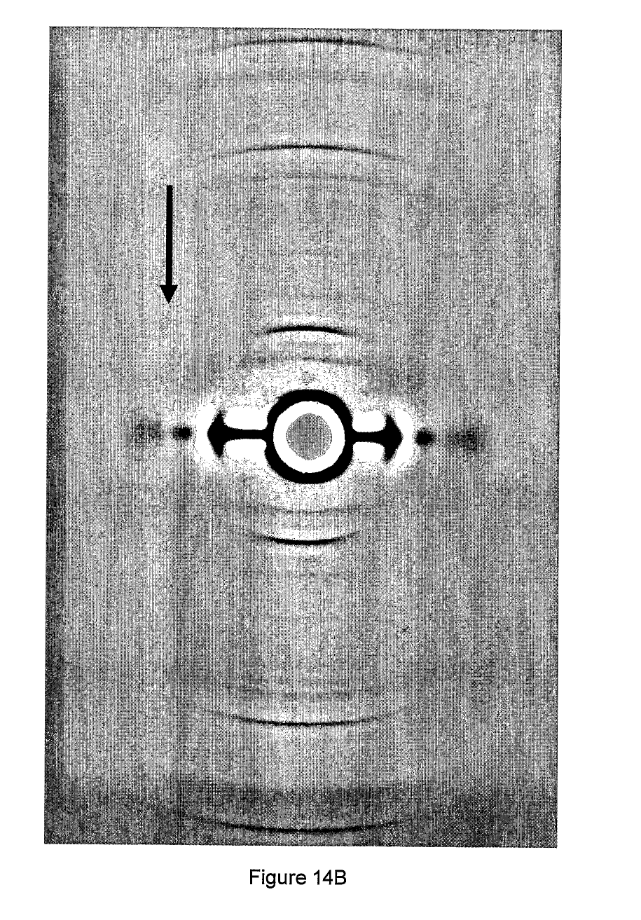

FIG. 14 shows the X-ray diffraction patterns of hair fibers from Tasmanian Devils (Sarcophilus laniarius). FIG. 14A is the diffraction pattern from a healthy animal while FIG. 14B is of the hair from a diseased animal. A difference can be noted in the equatorial region as marked by the arrow.

1. A method of analyzing a keratin sample from a subject so as to improve sensitivity and specificity of a diagnostic test for a pathological state in the subject comprising:
a) exposing the keratin sample to incident energy derived from an energy source;
b) receiving radiated energy from the keratin sample consequent upon impingement of the incident energy on the keratin sample;
c) passing at least a portion of the radiated energy received from the keratin sample through a transducer so as to derive subject specific data;
d) processing subject specific derived data;
e) comparing the processed subject specific data thus derived with a second group of reference data present in a reference database; wherein the second group of reference data is consistent with a presence of the pathological state in the subject; and
f) said processing method including the steps of applying an appropriate algorithm to said subject specific data prior to said step of comparing thereby to improve sensitivity and specificity relative to said reference data.

2. The method of claim 1 wherein said algorithm is applied to said reference data.

3. The method of claim 1 wherein said algorithm comprises a filtering operation.

4. The method of claim 3 wherein said algorithm comprises a median filtering operation using a defined pixel array.

5. The method of claim 4 wherein said pixel array is 3 by 3 pixel array.

6. The method of claim 1 wherein said step of filtering is followed by an averaging operation.

7. The method of claim 6 wherein said averaging operation is performed by using a 50 by 50 pixel array.

8. The method of claim 1 wherein said step of averaging is followed by a subtraction operation.

9. The method of claim 8 wherein said subtraction operation is performed by subtracting averaged data produced in claim 7 from the filtered data produced in claim 5.

10. The method of claim 1 wherein one dimensional intensity data is extracted from said subject specific data in order to determine spacing of relevant features.

11. The method of claim 10 wherein said intensity data along a single line starting from the centre of the image along a pre-selected plane.

12. The method of claim 10 wherein said intensity data is extracted by integrating sectors.

13. The method of analyzing a keratin sample as recited in claim 1 wherein the second group of data is correlated with the presence of the pathological state in the subject.

14. The method of analyzing a keratin sample as recited in claim 1 wherein the second group of data is indicative of the presence of the pathological state in the subject.

15. The method of analyzing a keratin sample as recited in claim 1 wherein the energy source is selected from a plurality of different energy sources.

16. The method of analyzing a keratin sample as recited in claim 1 wherein the keratin sample is selected from a plurality of different keratin samples.

17. The method of analyzing a keratin sample as recited in claim 1 wherein the second group of data is selected from a plurality of different groups of data.

18. The method of analyzing a keratin sample as recited in claim 1 wherein the derived data and the second group of data are analyzed using a plurality of different methods of comparison.

19. The method of analyzing a keratin sample as recited in claim 1 wherein at least a portion of the incident energy is absorbed by the keratin sample.

20. The method of analyzing a keratin sample as recited in claim 1 wherein, in use, the keratin sample can be obtained and analyzed in association with at least one of a pharmacy, a test kit, the subject's home, a health care clinic and a testing laboratory.

21. (canceled)

22. A method of analysis of the data derived in the method of claim 1 wherein said data is in the form of image data of an image derived from said transducer; said method of analysis comprising:
(a) extracting one-dimensional data along predetermined paths in said image so as to determine spacing of features in said image;
(b) defining substantially circular-oriented peak data about a centre point of said image from an analysis of said one-dimensional data; and
(c) applying intensity correction to said substantially circular-oriented peak data so as to better define said circular-oriented peak data as it appears in said image.
